(19) United States
(12) Patent Application Publication
(10) Pub. No.: US 2022/0280046 A1
(43) Pub. Date: Sep. 8, 2022

(54) SYSTEM AND METHOD FOR SCREENING ORTHOSTATIC HYPOTENSION BY USING HEART RATE-BASED MACHINE LEARNING ALGORITHM, AND WEARABLE MEASUREMENT DEVICE

(71) Applicant: TOBEDTX, Seoul (KR)

(72) Inventors: Byung Jo Kim, Seoul (KR); Jung Bin Kim, Seoul (KR)

(21) Appl. No.: 17/632,707

(22) PCT Filed: Aug. 6, 2020
(86) PCT No.: PCT/KR2020/010442

§ 371 Date: Feb. 3, 2022

(30) Foreign Application Priority Data
Aug. 7, 2019 (KR) 10-2019-0096268

(51) Int. Cl. A61B 5/021; A61B 5/024; A61B 5/00; G16H 50/20; G16H 50/30

(52) U.S. Cl. CPC A61B 5/021; A61B 5/024; A61B 5/6802; A61B 5/7267; A61B 5/7275; G16H 50/20; G16H 50/30

(57) ABSTRACT

An orthostatic hypotension screening system using a heart rate-based machine learning algorithm includes an input unit configured to receive a variable comprising at least one of a patient's age, blood pressure, an expiration (E)-inspiration (I) difference and an E:I ratio calculated from a heart rate, and a Valsalva ratio calculated according to a Valsalva method; and a determination unit configured to determine whether the patient has orthostatic hypotension according to a machine learning algorithm that is pre-trained based on the variable received through the input unit.

### TECHNICAL FIELD

The present application relates to an orthostatic hypotension screening system and method using a heart rate-based machine learning algorithm, and a wearing measuring device.

### BACKGROUND ART

Orthostatic intolerance (OI) refers to a disorder of the autonomic nervous system occurring when a patient stands up from a lying or sitting position and blood flow to the brain and heart is reduced, leading to lightheadedness, vertigo, blurred vision, palpitations, nausea, and fatigue.

Orthostatic hypotension (OH) is one of OIs and is defined as a condition in which there is no increase in a heart rate to maintain blood flow despite a fall in systolic blood pressure of 20 mmHg or more and diastolic blood pressure of 10 mmHg or more due to standing. Because OH may occur in a variety of disorders related to the autonomic nervous system such as Parkinson's disease, multiple atrophy, pure autonomic failure, and diabetic autonomic neuropathy, and is associated with an increased risk of falls, cardiovascular events, and cognitive impairment, it is necessary to early detect and timely manage OH.

A head-up tilt table test (HUT) is widely used to diagnose OH. However, there are many patients who are unable to maintain their posture on a tilt table due to physical disability, or have contraindications for the use of a HUT such as severe anemia, kidney or heart failure, heart valve disease, severe coronary artery disease, and acute and subacute stroke or myocardial infarction. In addition to these physical constrains, due to the burden of time and cost required to perform a HUT, and a high possibility of false-negative results caused by performing OH, which is generated by various stimuli in daily life, only within a set laboratory environment, there is a limit to repeatedly performing a HUT to monitor OH treatment response and symptom progression.

In order to overcome such limitations of a HUT, research is being conducted to discover alternative biomarkers for diagnosis of OH, but there is still no technology for timely and accurately diagnosing OH by using a change in a heart rate which may be obtained from non-postural stimuli without a HUT in daily life.

### DISCLOSURE OF THE INVENTION

### Technical Problem

Accordingly, in the art, there is a demand for a method of timely and accurately diagnosing orthostatic hypotension (OH) by using a change in a heart rate which may be obtained from non-postural stimuli without a head-up tilt table test (HUT) in daily life.

### Technical Solution

To solve the problems, an embodiment of the present disclosure provides an orthostatic hypotension (OH) screening system using a machine learning algorithm based on a heart rate measured from non-postural stimuli.

The orthostatic hypotension screening system using a heart rate-based machine learning algorithm may include: an input unit configured to receive a variable including at least one of a patient's age, blood pressure, an expiration (E)-inspiration (I) difference and an E:I ratio calculated from a heart rate, and a Valsalva ratio calculated according to a Valsalva method; and a determination unit configured to determine whether the patient has orthostatic hypotension according to a machine learning algorithm that is pre-trained based on the variable received through the input unit.

The orthostatic hypotension screening system using a heart rate-based machine learning algorithm may include: a wearable measuring device worn on a patient's body and configured to measure the patient's heart rate; a processing device configured to calculate an expiration (E)-inspiration (I) difference and an E:I ratio from the heart rate measured by the wearable measuring device, and calculate a Valsalva ratio from the heart rate measured by the wearable measuring device according to a Valsalva method; and a determination unit configured to determine whether the patient has orthostatic hypotension according to a machine learning algorithm that is pre-trained based on the E-I difference, the E:I ratio, and the Valsalva ratio calculated by the processing device.

Another embodiment of the present disclosure provides an orthostatic hypotension screening method using a heart rate-based machine learning algorithm.

The orthostatic hypotension screening method using a heart rate-based machine learning algorithm may include: calculating an expiration (E)-inspiration (I) difference and an E:I ratio from a patient's heart rate; calculating a Valsalva ratio according to a Valsalva method; and determining whether the patient has orthostatic hypotension according to a machine learning algorithm based on the E-I difference, the E:I ratio, and the Valsalva ratio.

Another embodiment of the present disclosure provides a wearable measuring device.

The wearable measuring device may be worn on a patient's body to measure the patient's heart rate, the wearable measuring device including software for executing the orthostatic hypotension screening method based on the heart rate-based machine learning algorithm and being configured to determine whether the patient has orthostatic hypotension by using the software based on the measured heart rate.

In addition, the solution to the problems does not list all features of the present disclosure. Various features and advantages and effects of the present disclosure will be understood in more detail with reference to the following specific embodiments

### Advantageous Effects

According to an embodiment of the present disclosure, orthostatic hypotension (OH) may be timely and accurately diagnosed by using a change in a heart rate which may be obtained from non-postural stimuli without a head-up tilt table test (HUT) in daily life.

### BRIEF DESCRIPTION OF THE DRAWINGS

FIG. 1 is a diagram illustrating a configuration of an orthostatic hypotension (OH) screening system using a heart rate-based machine learning algorithm, according to an embodiment of the present disclosure.

FIG. 2 is a diagram illustrating a configuration of an OH screening system using a heart rate-based machine learning algorithm, according to another embodiment of the present disclosure.

FIG. 3 is a flowchart illustrating an OH screening method using a heart rate-based machine learning algorithm, according to another embodiment of the present disclosure.

### BEST MODE FOR CARRYING OUT THE INVENTION

Hereinafter, embodiments of the present disclosure will be described in detail with reference to the attached drawings in order to enable one of ordinary skill in the art to embody and practice the present disclosure. While describing the present disclosure, detailed descriptions of related well-known functions or configurations that may blur the points of the present disclosure are omitted. Also, the same reference numerals denote elements having similar functions and actions throughout the drawings.

In addition, throughout the specification, when a portion is referred to as being “connected” to another portion, it may be “directly connected” or may be “indirectly connected” with intervening devices therebetween. When a portion “includes” an element, another element may be further included, rather than excluding the existence of the other element, unless otherwise described.

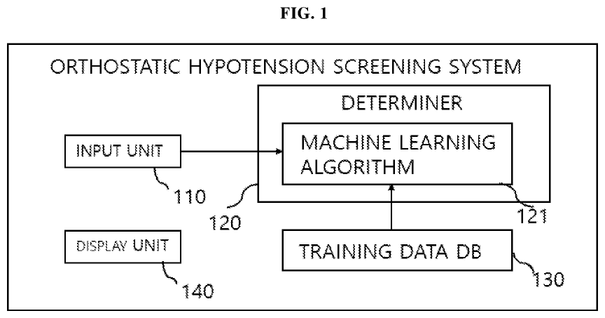

FIG. 1 is a diagram illustrating configuration of an orthostatic hypotension (OH) screening system using a heart rate-based machine learning algorithm, according to an embodiment of the present disclosure.

Referring to FIG. 1, an OH screening system 100 using a heart rate-based machine learning algorithm according to an embodiment of the present disclosure may include an input unit 110, a determination unit 120, and a training data database (DB) 130, and may further include a display unit 140

The input unit 110 is configured to receive a variable used to screen OH.

According to an embodiment, the input unit 110 may receive a variable including at least one of a patient's age and blood pressure, an E-I difference and an E:I ratio calculated from a heart rate, and a Valsalva ratio calculated according to a Valsalva method, and particularly, it is preferable that the received variable includes the E-I difference, the E:I ratio, and the Valsalva ratio. E represents expiration, and I represents inspiration. Also, the patient' heart rate may be measured during deep breathing, may be measured in a lying position at rest, or may be measured in a standing position at rest. That is, according to an embodiment of the present disclosure, whether the patient has OH may be determined based on a heart rate which may be obtained from non-postural stimuli including deep breathing, a lying position and a standing position at rest without a head-up tilt table test (HUT).

For example, the input unit 110 may receive the patient's age and blood pressure information corresponding to the patient's basic information from the patient's terminal, a medical staff terminal, or an external server (hospital information system). The blood pressure information may include reference systolic blood pressure information and diastolic blood pressure information.

Also, the input unit 110 may receive the E-I difference and the E:I ratio calculated from the heart rate measured during the non-postural stimuli, or may receive heart rate information measured during the non-postural stimuli and may calculate the E-I difference and the E:I ratio from the heart rate information. To this end, the input unit 110 may include a processing device for calculating the E-I difference and the E:I ratio.

In detail, when the heart rate measured during deep breathing according to an embodiment is used, a heart rate range may be measured during the deep breathing (e.g., six breaths per minute), and based on the heart rate range, the E-I difference may be calculated by subtracting a minimum heart rate during expiration from a maximum heart rate during inspiration for each of 6 breathing cycles, and the E:I ratio may be calculated as a ratio of a longest R-R interval during expiration to a shortest R-R interval during inspiration.

Also, the input unit 110 may receive the Valsalva ratio calculated according to a Valsalva method, or may receive heart rate information measured according to a Valsalva method and may calculate the Valsalva ratio from the heart rate information.

In detail, in a state where the patient is asked to blow through a mouthpiece attached to a manometer in a comfortable reclining posture according to the Valsalva method to maintain a pressure of 40 mmHg for 15 seconds, a heart rate rage may be measured, and the Valsalva ratio may be calculated by dividing a maximum R-R interval by a minimum R-R interval.

According to an embodiment, the measurement of a heart rate during deep breathing, at rest, or according to a Valsalva method may be performed by a wearable measuring device that may be worn on the patient's body, and to this end, the display unit 140 that is additionally provided may provide content that induces to perform deep breathing or Valsalva maneuver and a heart rate during the stimulus may be measured. Accordingly, the patient's heart rate may be measured during non-postural stimuli according to deep breathing, at rest, or a Valsalva method, and whether the patient has OH may be conveniently determined based on the patient's heart rate.

The determination unit 120 is configured to determine whether the patient has OH according to a machine learning algorithm 121 based on the variable received through the input unit 110.

According to an embodiment, the machine learning algorithm 121 may receive at least one of the patient's age, blood pressure, E-I difference, E:I ratio, and Valsalva ratio and may determine whether the patient has OH based on the same. In particular, it is preferable that the variable received by the machine learning algorithm 121 includes the E-I difference, the E:I ratio, and the Valsalva ratio.

To this end, the machine learning algorithm 121 may perform training in advance by using training data stored in the training data DB 130 that is pre-established. For example, the machine learning algorithm 121 may perform training to determine whether the patient has OH by using the training data stored in the training data DB 130, that is, age, blood pressure, E-I difference, E:I ratio, and Valsalva ratio data of patients diagnosed with OH by a HUT and non-OH patients.

According to an embodiment, the machine learning algorithm 121 may be a support-vector machine (SVM) algorithm, a K-nearest neighbor (KNN) algorithm, or a random forest algorithm, but the present disclosure is not limited thereto, and any of various training algorithms known to one of ordinary skill in the art may be selected and used.

Table 1 shows the performance according to a type of an algorithm used in a machine learning algorithm, and specifically, shows accuracy when an SVM algorithm, a KNN algorithm, and a random forest algorithm were applied by using five input variables including a patient's age, blood pressure, E-I difference, E:I ratio, and Valsalva ratio.

TABLE 1

| Algorithms | Precision | Recall | Accuracy |
|---|---|---|---|
| SVX | 0.83 | 0.88 | 0.84 |
| KNN | 0.83 | 0.88 | 0.84 |
| Random forest | 0.94 | 0.88 | 0.91 |

It is found from Table 1 that the accuracy when the random forest algorithm was applied was the highest.

When the machine learning algorithm 121 that performs training as described above is used, whether a patient has OH may be timely and accurately determined even in daily life based on a variable input in real time through the input unit 110.

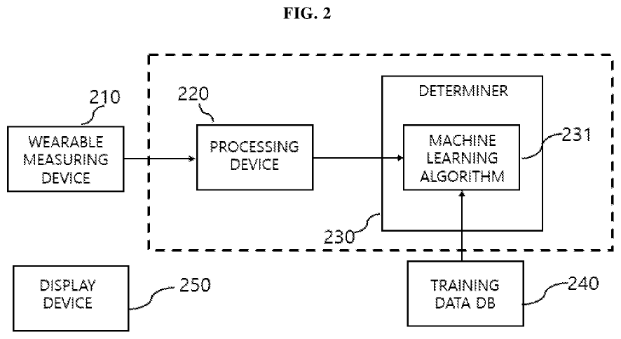

FIG. 2 is a diagram illustrating a configuration of an OH screening system using a heart rate-based machine learning algorithm, according to another embodiment of the present disclosure.

Referring to FIG. 2, an OH screening system 200 using a heart rate-based machine learning algorithm according to an embodiment of the present disclosure may include a wearable measuring device 210, a processing device 220, a determination unit 230, and a training data DB 240, and may further include a display device 250.

The wearable measuring device 210 may be worn on a patient's body and may measure the patient's heart rate.

The processing device 220 may calculate an E-I difference and an E:I ratio from the heart rate measured by the wearable measuring device 210, and may calculate a Valsalva ratio from the heart rate measured by the wearable measuring device 210 according to a Valsalva method.

The determination unit 230 may determine whether the patient has OH according to a machine learning algorithm 231 that is pre-trained based on the E-I difference, E:I ratio, and the Valsalva ratio calculated by the processing device 220.

The display device 250 may provide content that induces to perform depth breathing or Valsalva maneuver.

A specific function of each of elements of the OH screening system 200 using the heart rate-based machine learning algorithm of FIG. 2 is the same as that described with reference to FIG. 1, and thus, a repeated description thereof will be omitted.

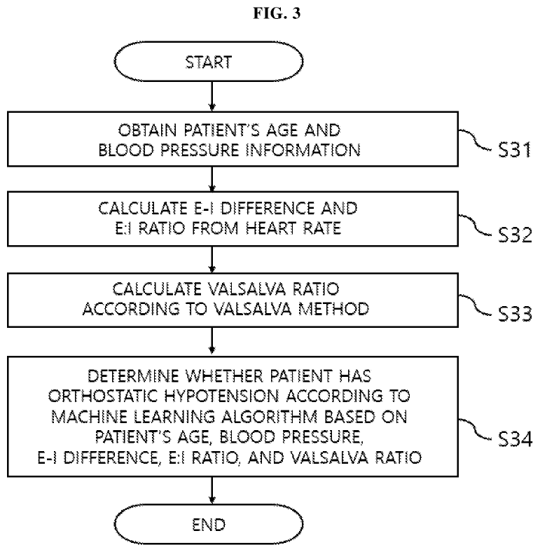

FIG. 3 is a flowchart illustrating an OH screening method using a heart rate-based machine learning algorithm, according to another embodiment of the present disclosure.

Referring to FIG. 3, first, a patient's age and blood pressure information may be obtained (S31), an E-I difference and an E:I ratio may be calculated from a measured heart rate (S32), and a Valsalva ratio may be calculated according to a Valsalva method (S33). The patient's heart rate may be measured during deep breathing, may be measured in a lying position at rest, or may be measured in a standing position at rest.

Although operations are sequentially performed in the order of operations S31 through S33 in FIG. 3, this is merely an example, the present disclosure is not limited thereto, and any order may be used as long as the patient's age, blood pressure, E-I difference, E:I ratio, and Valsalva ratio information may be obtained.

Next, whether the patient has OH may be determined according to a machine learning algorithm based on the information obtained in operations S31 through S33, that is, the patient's age, blood pressure, E-I difference, E:I ratio, and Valsalva ratio (S34).

A specific method for performing each operation of the OH screening method using the heart rate-based machine learning algorithm of FIG. 3 is the same as that described with reference to FIG. 1, and thus, a repeated description thereof will be omitted.

Also, the OH screening method using the heart rate-based machine learning algorithm of FIG. 3 may be performed by a processing device capable of executing the machine learning algorithm.

According to an embodiment of the present disclosure, a computer-readable storage medium storing instructions executable by a processor for performing each operation of the OH screening method using the heart rate-based machine learning algorithm of FIG. 3 may be provided.

According to another embodiment of the present disclosure, the OH screening method using the heart rate-based machine learning algorithm of FIG. 3 may be implemented as software, and the software may be mounted on a wearable measuring device that is worn on a patient's body to measure the patient's heart rate. Accordingly, the wearable measuring device may be used as an OH screening tool for measuring the patient's heart rate in daily life and determining whether the patient has OH based on the heart rate.

The present disclosure is not limited to the embodiments and the attached drawings. It will be apparent to one of ordinary skill in the art to which the present disclosure pertains that components according to the present disclosure may be substituted, modified, and changed without departing from the technical scope of the present disclosure.

1. An orthostatic hypotension screening system using a heart rate-based machine learning algorithm comprising:
an input unit configured to receive a variable comprising at least one of a patient's age, blood pressure, an expiration (E)-inspiration (I) difference and an E:I ratio calculated from a heart rate, and a Valsalva ratio calculated according to a Valsalva method; and
a determination unit configured to determine whether the patient has orthostatic hypotension according to a machine learning algorithm that is pre-trained based on the variable received through the input unit.

2. The orthostatic hypotension screening system of claim 1, wherein the heart rate of the patient is measured during deep breathing, measured in a lying position at rest, or measured in a standing position at rest.

3. The orthostatic hypotension screening system of claim 1, further comprising a display unit configured to provide content that induces to perform deep breathing or Valsalva maneuver.

4. An orthostatic hypotension screening system using a heart rate-based machine learning algorithm comprising:
a wearable measuring device worn on a patient's body and configured to measure the patient's heart rate;
a processing device configured to calculate an expiration (E)-inspiration (I) difference and an E:I ratio from the heart rate measured by the wearable measuring device, and calculate a Valsalva ratio from the heart rate measured by the wearable measuring device according to a Valsalva method; and
a determination unit configured to determine whether the patient has orthostatic hypotension according to a machine learning algorithm that is pre-trained based on the E-I difference, the E:I ratio, and the Valsalva ratio calculated by the processing device.

5. The orthostatic hypotension screening system of claim 4, further comprising a display device configured to provide content that induces to perform deep breathing or Valsalva maneuver.

6. An orthostatic hypotension screening method using a heart rate-based machine learning algorithm comprising:
calculating an expiration (E)-inspiration (I) difference and an E:I ratio from a patient's heart rate; calculating a Valsalva ratio according to a Valsalva method; and
determining whether the patient has orthostatic hypotension according to a machine learning algorithm based on the E-I difference, the E:I ratio, and the Valsalva ratio.

7. The orthostatic hypotension screening method of claim 6, further comprising obtaining the patient's age and blood pressure information, wherein the determining whether the patient has orthostatic hypotension comprises determining whether the patient has orthostatic hypotension by additionally considering the patient's age and blood pressure information.

8. A wearable measuring device worn on a patient's body to measure the patient's heart rate, the wearable measuring device comprising software for executing the orthostatic hypotension screening method based on the heart rate-based machine learning algorithm of claim 6 and being configured to determine whether the patient has orthostatic hypotension by using the software based on the measured heart rate
